(19) United States
(12) Patent Application Publication
(10) Pub. No.: US 2021/0085933 A1
(43) Pub. Date: Mar. 25, 2021

(54) MECHANICALLY ASSISTED INFLATION DEVICE HANDLE AND METHOD OF USE

(71) Applicant: Merit Medical Systems, Inc., South Jordan, UT (US)

(72) Inventor: Michael Dean Haslam, Sandy, UT (US)

(21) Appl. No.: 17/028,170

(22) Filed: Sep. 22, 2020

Related U.S. Application Data
(60) Provisional application No. 62/904,298, filed on Sep. 23, 2019.

(51) Int. Cl. A61M 25/10

(52) U.S. Cl. CPC A61M 25/10182; A61M 2205/073

(57) ABSTRACT

An inflation device including a handle mechanism configured to selectively engage and disengage threads within the device. In some instances, the threads are configured to couple a plunger to a syringe body. The handle mechanism may be configured to (1) provide a mechanical advantage and (2) change the location and direction of the input force.

### RELATED APPLICATIONS

This application claims priority to U.S. Provisional Application No. 62/904,298, filed on Sep. 23, 2019 and titled, “Mechanically Assisted Inflation Device Handle and Method of Use,” which is hereby incorporated by reference in its entirety.

### TECHNICAL FIELD

The present disclosure relates generally to devices used to pressurize, depressurize, or otherwise displace fluid, particularly in medical devices. More specifically, the present disclosure relates to devices used to pressurize, depressurize, or otherwise displace fluid along a line in order to inflate or deflate a medical device, such as a balloon.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an inflation device.

FIG. 2 is a cross-sectional view of the inflation device of FIG. 1 taken through plane 2-2 of FIG. 1.

FIG. 3 is an exploded view of the inflation device of FIG. 1.

FIG. 4 is an exploded view of a portion of the handle of the inflation device of FIG. 1.

FIG. 5 is a cross-sectional view of a portion of the inflation device of FIG. 1.

FIG. 6 is a cross-sectional view of the inflation device of FIG. 1 with fluid disposed in a portion of the device.

FIG. 7A is a detail view, taken through line 7A-7A of FIG. 2, of a portion of the handle of FIG. 1, in the configuration shown in FIG. 2.

FIG. 7B is a view of the detail portion, taken through line 7B-7B of FIG. 6, of the handle of FIG. 1, in the configuration shown in FIG. 6.

FIG. 8A is a cross-sectional view of the threaded portion of the inflation device of FIG. 1, in the configuration of FIGS. 2 and 7A.

FIG. 8B is the cross-sectional view of the threaded portion of the inflation device of FIG. 8A, in the configuration of FIGS. 6 and 7B.

FIG. 9 is a perspective view of the inflation device of FIG. 1 with fluid disposed within the device and a balloon coupled to the inflation device.

### DETAILED DESCRIPTION

An inflation device may include a syringe that utilizes threads to advance or retract a plunger by rotating the plunger handle relative to the body of the syringe such that the threads cause longitudinal displacement of the plunger relative to the body. In some instances, an inflation syringe may further include retractable threads, enabling a practitioner to disengage the threads and displace the plunger by simply pushing or pulling the plunger.

Certain inflation devices, such as those described in U.S. Pat. Nos. 5,047,015; 5,057,078; 5,163,904; and 5,209,732 include a mechanism in the handle of the device that allows the practitioner to disengage the threads by manipulating the mechanism. For example, in some instances the handle of such a device may include a “trigger” portion that may be configured to retract threads positioned on the plunger when the trigger is actuated.

An inflation device may further be configured such that the thread retraction mechanism includes elements that provide mechanical advantage, allowing a user to more easily manipulate the mechanism. Moreover, a mechanism may be configured to alter the location of an input force, which may provide flexibility and ease of operation to the device.

It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure but is merely representative of various embodiments. While the various aspects of the embodiments are presented in drawings, the drawings are not necessarily drawn to scale unless specifically indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the syringe portion of an inflation device, the proximal end of the syringe refers to the end nearest the handle and the distal end refers to the opposite end, the end nearest the inlet/outlet port of the syringe. Thus, if at one or more points in a procedure a physician changes the orientation of a syringe, as used herein, the term “proximal end” always refers to the handle end of the syringe (even if the distal end is temporarily closer to the physician).

“Fluid” is used in its broadest sense, to refer to any fluid, including both liquids and gasses as well as solutions, compounds, suspensions, etc., that generally behave as a fluid.

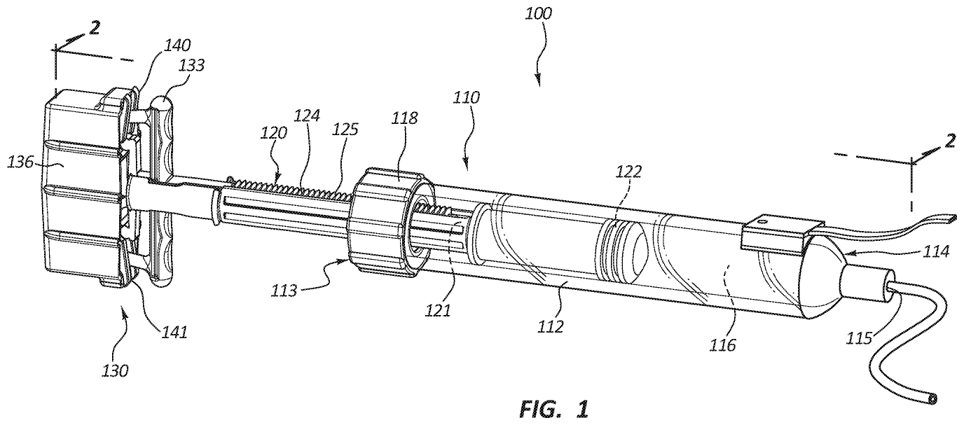

FIGS. 1-9 illustrate different views of an inflation device. In certain views the device may be coupled to, or shown with, additional components not included in every view. Further, in some views only selected components are illustrated, to provide detail into the relationship of the components. Some components may be shown in multiple views but not discussed in connection with every view. Disclosure provided in connection with any figure is relevant and applicable to disclosure provided in connection with any other figure.

FIG. 1 is a perspective view of an inflation device 100. In the illustrated embodiment, the inflation device 100 is partially comprised of a syringe 110. The inflation device 100 includes three broad groups of components; each group may have numerous subcomponents and parts. The three broad component groups are: a body component such as syringe body 112, a pressurization component such as plunger 120, and a handle 130.

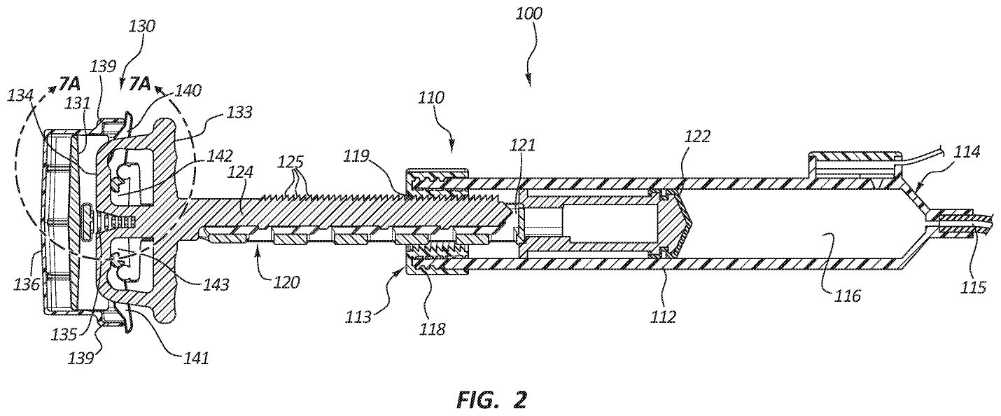

The syringe body 112 may be formed of a generally cylindrical hollow tube configured to receive the plunger 120. The syringe body 112 may include an inlet/outlet port 115 located adjacent a distal end 114 of the syringe body 112. In some embodiments, a nut 118 may be coupled to the syringe body 112 adjacent a proximal end 113 of the syringe body 112. The nut 118 may include a center hole configured to allow the plunger 120 to pass through the nut 118 into the syringe body 112. Further, the nut 118 may include internal nut threads 119 (FIG. 2) configured to selectively couple the nut 118 to the plunger 120 in some embodiments.

The plunger 120 may be configured to be longitudinally displaceable within the syringe body 112. The plunger 120 may be comprised of a plunger shaft 121 coupled to a plunger seal 122 at the distal end of the plunger shaft 121. The plunger shaft 121 may also be coupled to the handle 130 at the proximal end of the plunger shaft 121, the plunger shaft 121 spanning the distance between the plunger seal 122 and the handle 130.

The handle 130 broadly refers to the group of components coupled to the proximal end of the plunger 120, some of which may be configured to be graspable by a user. In certain embodiments, the handle 130 may be configured such that the user may manipulate the position of the plunger 120 by manipulating the handle 130. Further, in some embodiments the handle 130 may be an actuator mechanism, configured to manipulate components of the inflation device 100. In further embodiments, the actuator mechanism may include a lever mechanism.

Any and every component disclosed in connection with any of the exemplary handle configurations herein may be optional. That is, though the handle 130 broadly refers to the components coupled to the proximal end of the plunger shaft 121 that may be configured to be graspable by a user, use of the term “handle” is not meant to indicate that every disclosed handle component is always present. Rather, the term is used broadly, referring to the collection of components, but not specifically referring to or requiring the inclusion of any particular component. Likewise, other broad groupings of components disclosed herein, such as the syringe 110 or syringe body 112 and the plunger 120, may also refer to collections of individual subcomponents. Use of these terms should also be considered non-limiting, as each subcomponent may or may not be present in every embodiment.

As shown in FIG. 1, a fluid reservoir 116 may be defined by the space enclosed by the inside walls of the syringe body 112 between the plunger seal 122 and the distal end 114 of the syringe body 112. Accordingly, movement of the plunger seal 122 with respect to the syringe body 112 will alter the size and volume of the reservoir 116. Advancing the plunger 120 (displacing in the distal direction) may reduce the volume and/or increase the pressure within the syringe body 112. Similarly, retracting the plunger 120 may increase the volume and/or decrease the pressure within the syringe body 112.

As shown in FIGS. 1 and 2, in some embodiments, the syringe 110 may include a nut 118, coupled to the proximal end 113 of the syringe body 112. The nut 118 may utilize threads or other coupling mechanisms to couple the nut 118 to the syringe body 112. The nut 118 may additionally include internal nut threads 119 configured to couple the nut 118 to a portion of the plunger 120. The plunger 120 may also include external plunger threads 125 configured to couple the plunger 120 to the nut 118. The plunger 120 may thus be translated longitudinally with respect to the syringe body 112 by rotating the plunger 120 such that the interaction of the nut threads 119 and the plunger threads 125 results in the longitudinal translation of the plunger 120. Thus, when the plunger threads 125 and the nut threads 119 are engaged, movement of the plunger 120 is constrained with respect to the syringe body 112, though the plunger 120 is not necessarily fixed with respect to the syringe body 112. For example, the plunger 120 may be rotatable, but not directly translatable, when the threads 125, 119 are engaged.

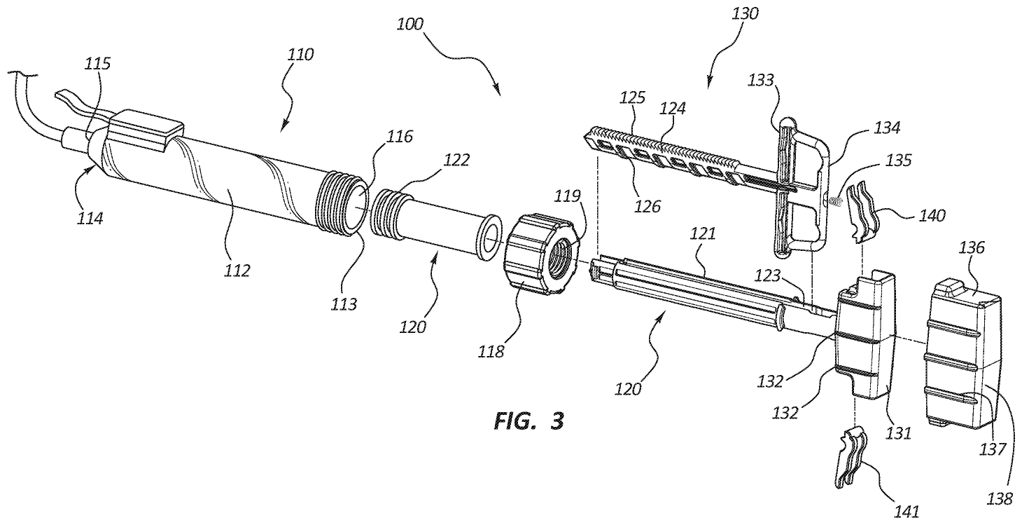

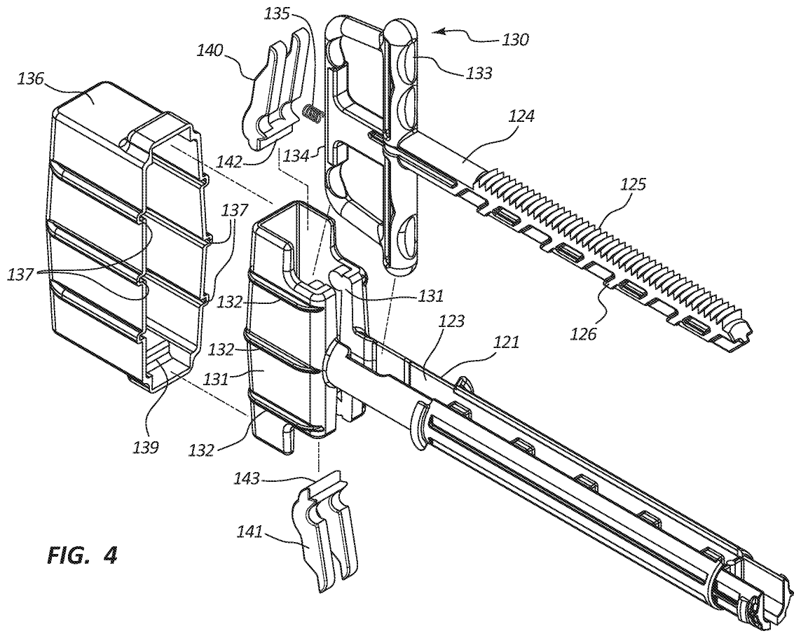

The plunger threads 125 may be configured such that they may be retracted within the plunger shaft 121. As shown in FIGS. 3 and 4, in some embodiments, the plunger threads 125 do not extend 360 degrees around the axis of the plunger shaft 121. Furthermore, as shown in FIGS. 1-4, the plunger threads 125 may be formed on a thread rail 124 that may be disposed within a groove 123 in the plunger shaft 121.

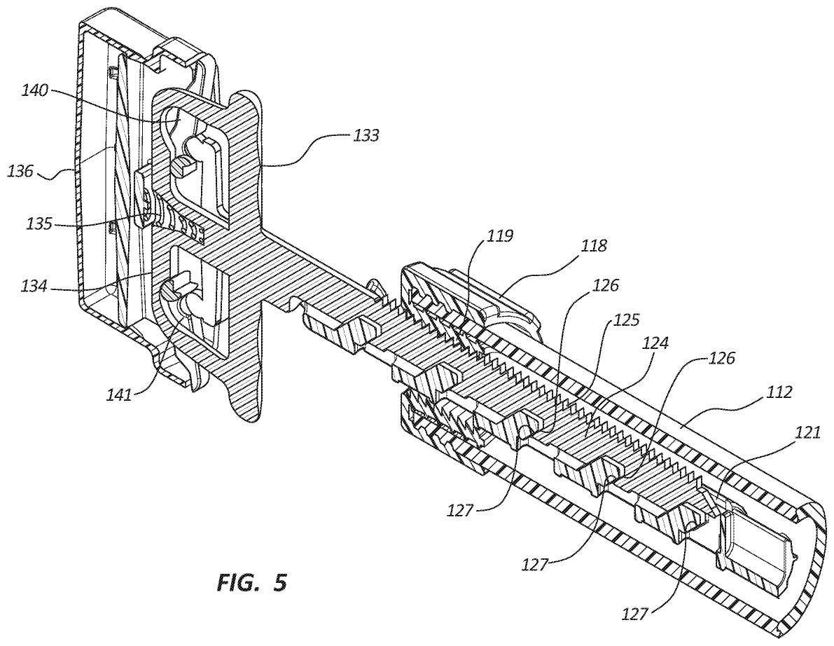

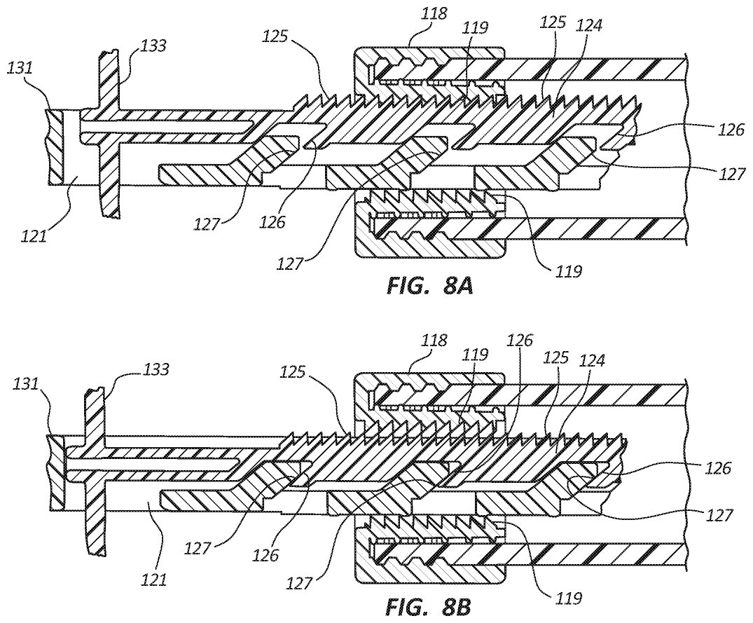

The thread rail 124 may be configured such that interaction between angled surfaces 126 on the thread rail 124 and the angled surfaces 127 (FIG. 5) within the groove 123 interact such that the plunger threads 125 may be retractable within the plunger shaft 121. The relationship between the angled surfaces 126 on the thread rail 124 and the angled surfaces 127 within the groove 123 (FIG. 4) is shown in FIGS. 5, 8A, and 8B. Translation of the thread rail 124 in the proximal direction simultaneously causes the thread rail 124 to retract toward the center axis of the plunger shaft 121 due to the interaction of the angled surfaces 126 on the thread rail 124 with the angled surfaces 127 in the groove 123. Similarly, translation of the thread rail 124 in the proximal direction causes the thread rail 124 to move away from the center axis of the plunger shaft 121. In the illustrated embodiment, a distally oriented biasing force acting on the thread rail 124 may bias the plunger threads 125 to the non-retracted position. As such, a singular proximally oriented force applied to the handle 130 (specifically the trigger member 133) may decouple the threads 125, 119. It will be appreciated by one of ordinary skill in the art having the benefit of this disclosure that it is within the scope of this disclosure to modify the angles and interfaces such that a distally oriented biasing force on the thread rail 124 would bias the plunger threads 125 in the retracted position. As mentioned above, analogous mechanisms are disclosed in U.S. Pat. Nos. 5,047,015; 5,057,078; 5,163,904; and 5,209,732.

FIGS. 8A and 8B illustrate two positions of the thread rail 124 with respect to the internal nut threads 119 and the plunger shaft 121. FIG. 8A shows thread rail 124 disposed in a non-retracted position, such that the plunger threads 125 are engaged with the internal nut threads 119. FIG. 8B shows the thread rail 124 sufficiently retracted into the plunger shaft 121 such that the plunger threads 125 are not engaged with the internal nut threads 119.

Embodiments that utilize retractable threads may allow a user to displace the plunger shaft 121 relative to the syringe body 112 either through rotation of the plunger shaft 121 (and the subsequent interaction of threads), or by retracting the plunger threads 125 and displacing the plunger shaft 121 by applying opposing forces on the plunger shaft 121 and the syringe body 112. (The forces, in turn, may move the plunger shaft 121 distally or proximally with respect to the syringe body 112.) Both methods of displacement may be utilized during the course of a single therapy.

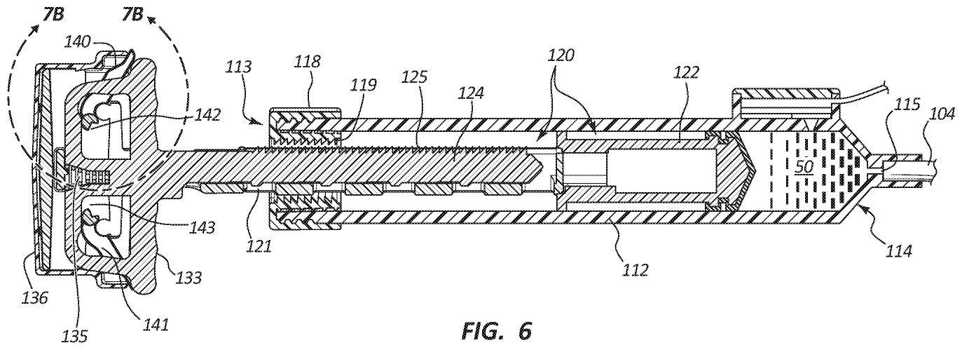

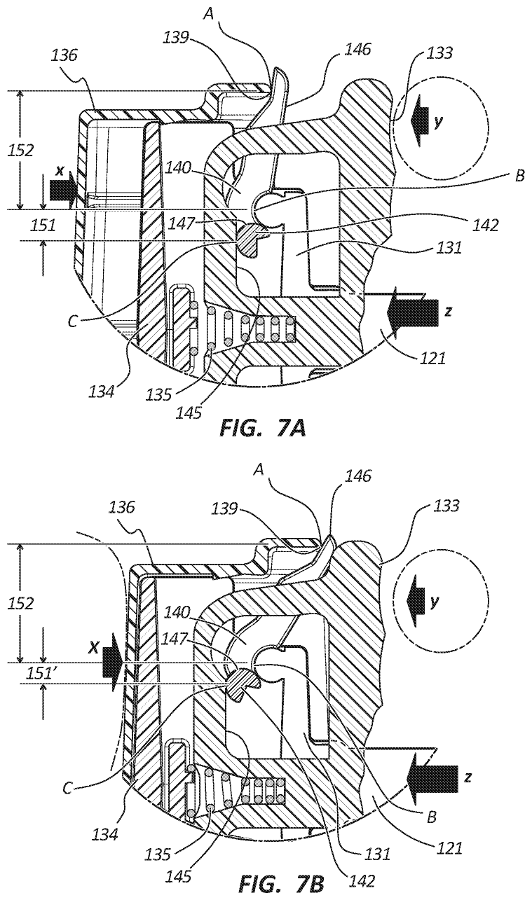

FIG. 6 is a cross-sectional view of the inflation device of FIG. 1 with fluid 50 disposed within the reservoir 116. FIGS. 2 and 6 illustrate the inflation device of FIG. 1 in a first configuration, with the handle released and the threads engaged (FIG. 2) and a second configuration, with the handle actuated and the threads disengaged (FIG. 6). These configurations are also shown in additional detail in FIGS. 7A-8B as described below. When comparing these configurations, it may be noted that, in the illustrated embodiment, when the handle is actuated and the threads disengaged, the trigger member 133 is laterally displaced (as well as axially displaced), as shown in FIG. 6 as compared to FIG. 2. With continued reference to FIG. 6, in some instances, a practitioner may desire to quickly displace the plunger shaft 121, for instance, while priming the inflation device or while priming or deflating an attached medical device such as a balloon. Quick displacement of the plunger shaft 121 may be accomplished by retracting the plunger threads 125 and sliding the plunger shaft 121 relative to the syringe body 112. For example, a practitioner may quickly fill the reservoir 116 with the fluid 50 by disengaging the plunger threads 125 and pulling the plunger shaft 121 in a proximal direction with respect to the syringe body 112. Further, a practitioner may quickly force the fluid 50 into lines leading to other devices or quickly expel unwanted air bubbles from the reservoir 116 by retracting the plunger threads 125 and advancing the plunger shaft 121.

In other instances, the practitioner may desire more precise control over the position of the plunger shaft 121 (for example when displacing the plunger shaft 121 in order to adjust the fluid pressure within the reservoir 116) or it may simply be difficult or impossible without a mechanical advantage to displace the plunger shaft 121 due to high fluid pressure within the reservoir 116. In these instances, the practitioner may opt to displace the plunger shaft 121 by rotation of the plunger shaft 121.

Referring back to FIG. 4, the handle 130 of the inflation device 100 (FIG. 1) may include components that enable a practitioner to retract the thread rail 124 of the plunger 120. In some embodiments, the plunger shaft 121 may be fixed to a first member such as the inner member 131 of the handle 130. The thread rail 124 may be fixed to a trigger 133 component of the handle 130. Further, a biasing component 135 may be configured to bias the trigger 133 in a distal direction relative to the plunger shaft 121. Because the trigger 133 is fixed to the thread rail 124, a distally oriented force on the trigger 133 will result in a distally oriented force on the thread rail 124 as well. The force provided by the biasing component 135 (hereafter also referred to as the biasing force) may thus bias the thread rail 124 in a non-retracted position as described above. Conversely, overcoming the biasing force and translating the trigger 133 in a proximal direction with respect to the plunger shaft 121 and the inner member 131 may retract the plunger threads 125. In some embodiments, the biasing force 135 may be greater than a force required to proximally displace the plunger 130 within the syringe body 112.

In some embodiments, the handle 130 may further include a second member such as the outer sleeve 136 and one or more levers 140, 141. The levers 140, 141 may be disposed such that they provide mechanical advantage, enabling the user to more easily overcome the biasing force and displace the trigger 133 toward the inner member 131.

Referring particularly to FIGS. 4, 6, 7A, and 7B, portions of the handle 130 that interact with the lever 140 may be the mirror image of the portions of the handle 130 that interact with the lever 141. Thus, in some embodiments, disclosure provided in connection with one lever is equally applicable to the other lever. Furthermore, it is within the scope of this disclosure to include levers on each side of the handle that are not identical or to include a single lever.

FIG. 7A is a detail view of a portion of the handle of the inflation device, in the configuration shown in FIG. 2. FIG. 7B is a detail view of the same portion of the handle of the inflation device, in the configuration shown in FIG. 6. As shown in the detail views of FIGS. 7A and 7B, the outer sleeve 136 contacts a first lever arm 146 of the lever 140 at point A. The outer sleeve 136 may include a first lever contact surface 139 configured to contact the first lever arm 146. A distally oriented force manually applied to the outer sleeve 136 will thus exert a distally oriented force on the first lever arm 146 at point A through contact of the first lever contact surface 139 with the first lever arm 146. The lever 140 may be coupled to the plunger shaft 121 via pivot point B. A cross bar 142 disposed on a second lever arm 147 of the lever 140 may thus exert a proximally oriented force on a second lever contact surface 145 included on a top member 134 of the trigger 133 at point C. Thus, a manually applied force that acts distally on the outer sleeve 136 is transferred by the levers 140, 141 and results in the cross bars 142, 143 applying a proximal force on the trigger 133. As discussed above, in the illustrated embodiment, a proximal force on the trigger 133 causes the thread rail 124 to retract, disposing the plunger 120 in a decoupled state.

It is within the scope of this disclosure to alter the shape or form of the levers 140, 141. For instance, the lever 140 is shown with an inside radius near the pivot point B that mates with an outside radius formed on a portion of the inner member 131. It is within the scope of this disclosure to alter the design such that the outside radius is formed on the lever 140 and the inside radius is formed on the inner member 131. The first lever arm 146 and the second lever arm 147 may also be curved or angled in one or more directions. Similar design modifications to the levers or any other component are equally within the scope of this disclosure. In the illustrated embodiment, the length of the first lever arm 146 is greater than the length of the second lever arm 147, meaning the distance from the pivot point B to the end of the first lever arm 146 is greater than the distance from the pivot point B to the end of the second lever arm 146. In other embodiments, the design could be modified such that the length of the second lever arm 147 is greater than the length of the first lever arm 146. Moreover, the levers 140, 141 may be modified such that the pivot point B is located at one end of each lever, rather than the pivot point located between the force transferring contact points A, C as in the illustrated embodiment. Furthermore, any combination of these alternative designs is within the scope of this disclosure, including designs where each of two levers has a different design, the handle includes a single lever, or compliant mechanisms are utilized to transfer force and/or provide mechanical advantage.

FIG. 7A illustrates the lever mechanism of the handle 130 with the plunger (120 of FIG. 2) in a coupled state, i.e., when the plunger 120 is coupled to the syringe body 112 via the threads 125, 119. As noted above, in the illustrated embodiment, this configuration correlates to the configuration wherein the handle 130 is released and the threads 125, 119 are engaged. In the configuration of FIG. 7A, external forces are not constraining or compressing the trigger 133 or the outer sleeve 136. (As discussed below, FIG. 7A does include indicia showing where forces may be applied to actuate the handle to displace the elements into the configuration of FIGS. 6, 7B and 8B.)

FIG. 7A also indicates a transverse distance (perpendicular to a longitudinal axis of the plunger 120) between point A and point B defining a first moment arm length 152. Also shown is a transverse distance between point C and point B defining a second moment arm length 151. As can be seen in the figure, these moment arms correlate with the first lever arm 146 and second lever arm 147. In the illustrated embodiment, the first moment arm length 152 is longer than the second moment arm length 151. In other embodiments, the first moment arm length 152 may be equal to or shorter than the second moment arm length 151. In the illustrated embodiment, a distal displacement distance of the outer sleeve 136 with respect to the plunger shaft 121 is thus converted by the lever 140 to a shorter proximal displacement distance of the trigger 133 with respect to the plunger shaft 121, creating a mechanical advantage (due to the difference between the first moment length 152 and the second moment arm length 151). The mechanical advantage may thus be understood as converting a force X applied to the outer sleeve 136 in a distal direction into a proximally applied force acting on the thread rail (124 of FIG. 5) via the trigger 133. In other words, application of force X may result in a force, acting on point C, that tends to displace the trigger 133 in the same manner as a force applied directly to the trigger 133, such as that illustrated as force Y. (As discussed below, in certain uses, forces X and Y may also be simultaneously applied by external elements, e.g. portions of a practitioner's hand, though force Y would be supplemented by the force exerted at point C due to application of force X). Force on the thread rail 124 (whether due to application at point C due to input by force X alone, or by a combination of forces X and Y) may thus be directed to counter and overcome the force exerted by the biasing member 135, compress the biasing member 135, and displace the thread rail 124. Hence, the lever 140 provides a mechanical advantage in decoupling the plunger 120 from the syringe body 112 when a single force is externally applied to the handle 130 in a distal direction.

Furthermore, application of distal force X results in a reactionary force Z (assuming that the inflation device 100 is constrained such that force X does not simply displace the entire inflation device 100). In some instances, a proximal force manually applied to the syringe body 112 in opposition to the manually applied distal force to the outer sleeve 136 may be transferred to the plunger shaft 121 when the thread rail 124 is engaged, and result in at least a portion of the reactionary force Z. When force X is sufficient to compress the biasing member 135 and displace the thread rail 124, reactionary force Z will no longer have a component supplied by engagement of the thread rail 124 with the syringe body 112. At that point, the reaction force Z may only result from the friction force between the plunger seal 122 and the syringe body 112 (assuming there is no pressure in the reservoir of the syringe body 112). Accordingly, when force X is also sufficient to overcome force Z (as supplied by such friction) the plunger 122 may be advanced within the syringe body 112 due to application of force X. However, when force Z is only supplied by such friction, force Z may not be sufficient to compress the biasing member 135, resulting in expansion of the biasing member 135, displacement of the thread rail 124 in a distal direction, and, thus, reengagement of the thread rail 124 with the syringe body 112. This reengagement again allows force on the syringe body 112 to be transferred to the plunger shaft 121, such that force Z again has a component supplied by forces exerted on the syringe body 112. This, in turn, may increase force Z, again compressing the biasing member 135, and cause displacement and retraction of the thread rail 124. Hence, advancement of the plunger 120 in response to a distally oriented force applied to the handle 130 (absent a proximal force externally applied to the trigger 133) may result in repeated disengagement and re-engagement of the thread rail 124 as the plunger 120 is advanced, causing a discontinuous pattern of engagement/disengagement and a “rough” feel or sound as the threads repeatedly engage/disengage. As further detailed below, in some embodiments, the lever mechanism may be configured to inhibit re-engagement of the thread rail 124 during advancement of the plunger 120 when the distally oriented force is manually applied to the handle 130 absent a proximal force manually applied to the trigger 133.

In some embodiments, the mechanical advantage may be configured (due to lever 140 size, relative displacement of the outer sleeve 136 and trigger 133, stiffness of the biasing member 135, and the ratio of the first moment arm length 152 over the second moment arm length 151) such that a distally directed force X on the outer sleeve 136 to decouple the plunger 120 is less than the friction force between the plunger seal 122 and the syringe body 112. In other words, the inflation device 100 may be configured such that the magnitude of the distally directed force X applied on the outer sleeve 136 required to decouple the plunger 120 from the syringe body 112 is less than the force required to advance the plunger 120 after the plunger 120 is decoupled from the syringe body 112. In such embodiments, frictional resistance to advancement of the plunger 120 is thus sufficient to keep the biasing member 135 compressed such that the threads do not discontinuously engage/disengage as the plunger 120 is advanced due to application of force X. Such embodiments may be configured such that application of force X allows for smooth and/or continuous advancement of the plunger 120 without application of an external force on the trigger 133 (such as force Y). In this configuration, the handle mechanism may thus supply the mechanical advantage at a first magnitude or supply a first factor of the mechanical advantage. As detailed below, during actuation of the handle, the magnitude of the mechanical advantage may change.

FIG. 7B illustrates the lever mechanism of the handle 130 with the plunger 120 in a decoupled state, i.e., when the plunger 120 is decoupled from the syringe body 112 due to actuation of the handle and retraction of the thread rail 124. In this configuration, the outer sleeve 136 is displaced distally relative to the position shown in FIG. 7A and the trigger 133 is displaced proximally relative to the position shown in FIG. 7A. As noted above, displacement of the trigger 133 may be due to external application of force X (and the transfer of that force at point C), external application of force Y, or a combination thereof. In the illustrated configuration, point C of lever arm 147 is displaced in the transverse direction toward the pivot point B. As such, the second moment arm length 151′ in FIG. 7B is shorter than the second moment arm length 151 in FIG. 7A, resulting in a second magnitude for the mechanical advantage greater than the first magnitude discussed in connection with FIG. 7A. In other words, the magnitude of the mechanical advantage in the configuration shown in FIG. 7B (the second factor of the mechanical advantage, or the mechanical advantage generated by the configuration of FIG. 7B) may be greater than the magnitude of the mechanical advantage in the configuration shown in FIG. 7A (the first factor of the mechanical advantage, or the mechanical advantage generated by the configuration of FIG. 7A). As such, the required amount of distally directed force X on the outer sleeve 136 to overcome the biasing force in opposition to the reaction force Z is greater when the plunger 120 is in the coupled state than when the plunger 120 is in the decoupled state. Hence, the lever mechanism provides for a lower required amount of distally directed force X to be applied to the outer sleeve 136, to maintain the plunger 120 in the decoupled state after the plunger 120 is decoupled from the syringe body 112. Said another way, the amount of externally applied distal force X on the outer sleeve 136 required to distally displace the outer sleeve 136 with respect to the plunger shaft 121 at a first position of the outer sleeve 136 may be greater than the amount of manually applied distal force X on the outer sleeve 136 required to distally displace the outer sleeve 136 with respect to the plunger shaft 121 at a second position of the outer sleeve 136 wherein the second position of the outer sleeve 136 is distal of the first position. In some embodiments, the second factor of the mechanical advantage (in combination with the biasing force) may provide for a lower amount of distally directed force X (applied to the outer sleeve 136) required to maintain decoupling of the plunger 120 when the plunger 120 is being displaced within the syringe body 112. As the amount of force X in such instances is less than the friction force between the plunger seal 122 and the syringe body 112, the plunger may be advanced in a continuous manner (without engaging/disengaging threads) when a distally oriented force is externally applied to the outer sleeve 136. Again, this may be stated as, in accordance with the second factor of the mechanical advantage, a distally directed manual force X on the sleeve 136 required to maintain decoupling of the plunger 120 from the syringe body 112 may be less than a distally directed manual force X on the sleeve 136 required to distally displace the plunger 120 within the syringe body 112 to decouple the plunger 120 from the syringe body 112.

In some embodiments, the friction force between the plunger seal 122 and the syringe body 112 may at least partially define the reaction force Z on the plunger shaft 121. In some embodiments, the friction force may substantially define the complete reaction force Z on the plunger shaft 121. Further, the friction force may be different when the plunger 120 is stationary with respect to the syringe body 112 than when the plunger 120 is moving. In other words, the static friction force between the plunger seal 122 and the syringe body 112 may be different than the dynamic friction force. In some instances, the dynamic friction force may be less than the static friction force. In some embodiments, the first factor of the mechanical advantage may provide for a single required force X (required to decouple the plunger 120) externally applied to the handle in the distal direction to be less than the dynamic friction force. In other embodiments, the first factor of the mechanical advantage may provide for the single required force X externally applied to the handle to be less than the static friction force and greater than the dynamic friction force. Similarly, the second factor of the mechanical advantage may provide for the single required force X to be less than the dynamic friction force. In some embodiments, a pressure within the syringe body 112 may also at least partially define the reaction force Z on the plunger shaft 121.

It will be appreciated by one of ordinary skill in the art having the benefit of this disclosure that, in many instances, a proximal force may be manually applied to the trigger 133 at the same time a distal force is manually applied to the outer sleeve 136. For example, when the handle 130 is grasped by a user, the user may actuate the handle 130 by squeezing the trigger 133 with his or her fingers. This action may coincide with a distally oriented force exerted on the outer sleeve 136 by the palm of the user's hand. Accordingly, the forces applied in this manner may be understood as a proximal force on the trigger 133 and a distal force on the outer sleeve 136. The mechanism of the levers 140, 141 converts the distally oriented force exerted on the outer sleeve 136 in combination with the manually applied proximal force on the trigger 133 into a combined proximal force on the trigger 133 to overcome the biasing force and retract the thread rail 124. In such an instance, the combination of the manually applied distal force on the outer sleeve 136 and the manually applied proximal force on the trigger 133 may also provide a mechanical advantage in decoupling the plunger 120 from the syringe body 112.

In the illustrated embodiment, a single force applied to the handle 130 in the distal direction exceeding a first specified amount may decouple the plunger 120 from the syringe body 112. A single force applied to the handle 130 in the distal direction exceeding a second specified amount may maintain decoupling the plunger 120 from the syringe body 112. A single force applied to the handle 130 in the distal direction exceeding a third specified amount may overcome a static friction between the plunger seal 122 and the syringe body 112 and initiate advancement of the plunger 120. A single force applied to the handle 130 in the distal direction exceeding a fourth specified amount may overcome a dynamic friction between the plunger seal 122 and the syringe body 112 and maintain advancement of the plunger 120. The first specified amount may be greater than the second specified amount and less than the third specified amount and/or the fourth specified amount. The second specified amount may be less than the third specified amount and/or the fourth specified amount.

In some embodiments, friction forces may further inhibit re-engagement of the thread rail 124. For example, a friction force between the first lever contact surface 139 and the first lever arm 146 at point A and/or a friction force between the cross bar 142 and the second lever contact surface 145 at point C may provide for a force X required to prevent proximal displacement of the outer sleeve 136 away from the position as shown in FIG. 7B to be less, and in some instances substantially less, than a force X required to distally displace the sleeve into the position as shown in FIG. 7B. As such, the friction force between the first lever contact surface 139 and the first lever arm 146 at point A and/or the friction force between the cross bar 142 and the second lever contact surface 145 at point C may further inhibit re-engagement of the thread rail 124 during advancement of the plunger 120 when the distally oriented force is manually applied to the handle 130 absent a proximal force manually applied to the trigger 133.

In the illustrated embodiment, a single force applied to the outer sleeve 136 in the distal direction may be transferred to the plunger shaft 121 indirectly via the lever mechanism and the biasing component 135. In some instances, the single force applied to the outer sleeve 136 in the distal direction may distally displace the outer sleeve 136 relative to the plunger shaft 121 such that the outer sleeve 136 bottoms-out on the plunger shaft 121 and the single force is transferred rigidly to the plunger shaft 121.

A handle configured to provide a mechanical advantage when retracting a thread rail may be desirable for certain therapies that require large syringes or high pressure. Such therapies may also require a larger biasing force due to the size of the device or the pressure within the device. A handle providing a mechanical advantage may make devices configured for such therapies easier to use.

As described above, and illustrated in the figures, in some embodiments, the levers 140, 141 may not be pinned or otherwise mechanically coupled to any of the other parts. In some embodiments, the levers 140, 141 may be only be constrained due to contact with other components of the device. Likewise, the outer sleeve 136 may not be mechanically fastened to any other component, though—like the levers 140, 141—contact between portions of the outer sleeve 136 and other components may be utilized to secure the position of the outer sleeve 136 with respect to the other components. Thus, in some embodiments the levers 140, 141 and the outer sleeve 136 may be allowed to “float” with respect to the other parts. A floating assembly as described above may allow certain components multiple degrees of freedom with respect to the other parts. For example, as explained below, in some embodiments the trigger 133 may be displaced in both the longitudinal and transverse directions (with respect to the outer sleeve 136) when the trigger 133 is actuated.

As shown in FIGS. 3 and 4, the outer sleeve 136 may also include slots 137 configured to mate with ridges 132 formed on the outer surface of the inner member 131. The interaction between these slots 137 and ridges 132 constrains the movement of the outer sleeve 136 with respect to the inner member 131; that is, the two components may only travel (with respect to each other) in a single direction, parallel to the longitudinal axis of the syringe body 112. As mentioned above, in the illustrated embodiment, the trigger 133 travels in a direction transverse to the longitudinal axis of the syringe body 112 (in addition to travel along the longitudinal axis) when it is compressed, due to the interaction of the angled surfaces 126, 127 of the thread rail 124 and the plunger shaft 121. Ridges and slots, such as those of the illustrated embodiment (132, 137), may provide a degree of usability and comfort to the device, as the portion of the outer sleeve 136—which may be in contact with the palm of the user in some instances—does not slide in a transverse direction.

Many design modifications relating to the outer sleeve 136 are within the scope of the current disclosure. For example, in the illustrated embodiments, the outer sleeve 136 has a cap-like shape, fitting over the inner member 131. In other embodiments, the outer sleeve 136 may instead be designed as a button that slides into the inner member 131 when it is compressed. Likewise, any other longitudinally actuatable component may be utilized in place of the outer sleeve 136.

The handle mechanism described above, and shown in each of FIGS. 1-9, may also be utilized to change the location and direction of an input force required to retract the plunger threads 125. The mechanism may allow a user to draw the trigger 133 toward the inner member 131 (and thus retract the plunger threads 125) solely by applying a distally oriented force to a top surface 138 of the outer sleeve 136. As outlined above, the levers 140, 141 transfer this force to the trigger 133, which retracts the plunger threads 125.

In some instances, a user such as a medical practitioner, may desire to displace the plunger 120 in a distal direction with only one hand. This may be accomplished by grasping the syringe body 112 and using a surface, for example a table top, to apply a distally oriented force on the top surface 138 of the outer sleeve 136. In this manner, a mechanism such as that described above may enable a practitioner to displace the plunger in a one-handed fashion.

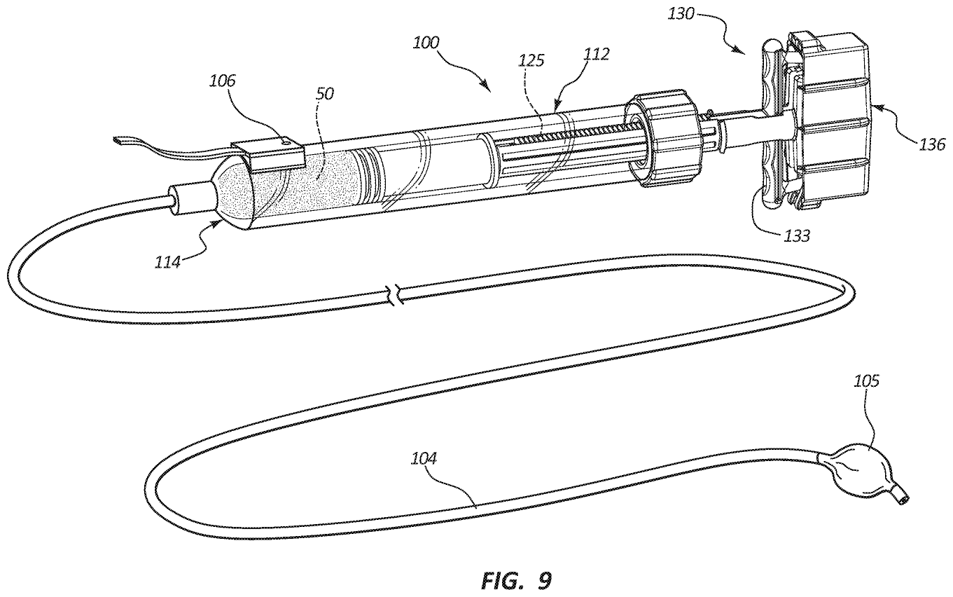

FIG. 9 is a perspective view of the inflation device 100 of FIG. 1 with fluid 50 disposed within the device and a balloon 105 coupled to the inflation device 100 via a delivery line 104. Referring now to components shown in FIG. 9 as well as the other figures, in some instances it may be desirable to operate the syringe 110 “one-handed” as described above in order to prime the system. For example, a practitioner may utilize the inflation device 100 in connection with a therapy that includes the balloon 105, such as an angioplasty. The practitioner may initially fill the syringe body 112 with the fluid 50, such as a contrast fluid, by drawing the plunger 120 back in the proximal direction. In some instances, the practitioner will do so by grasping the handle 130 of the inflation device 100 with a first hand, while grasping the syringe body 112 with a second hand. The practitioner may then retract the plunger threads 125 by squeezing the trigger 133 and the outer sleeve 136 together with his or her first hand, then drawing the plunger 120 back in the proximal direction.

After a desired amount of fluid 50 is disposed within the syringe body 112, the practitioner may orient the syringe body 112 such that the distal end 114 of the syringe body 112 is above the handle 130, so any air bubbles in the fluid 50 will tend to rise to the distal end 114 of the syringe body 112. The practitioner may also shake, tap, or otherwise disturb the syringe 110 in order to facilitate movement of any air bubbles in the fluid 50. The practitioner may then prime the syringe 110 by displacing the plunger 120 in a distal direction with respect to the syringe body 112, thereby forcing the air bubbles from the syringe body 112.

In some instances, the practitioner will displace the plunger 120 as described after first retracting the plunger threads 125. This may be accomplished in any manner disclosed herein, including the one-handed operation described above. That is, the practitioner may prime the inflation device 100 simply by grasping the syringe body 112 with one hand and using a static object or surface, such as a table top, to exert a distally directed force on the top surface 138 of the outer sleeve 136. The force on the outer sleeve 136 will both (1) retract the plunger threads 125 via the handle 130 mechanism and (2) act to displace the plunger 120 in a distal direction with respect to the syringe body 112. This orientation positions the syringe body 112 in a potentially desirable position to allow air to travel to the distal end 114 of the syringe body 112 while simultaneously orienting the handle 130 such that the top surface 138 of the outer sleeve 136 directly faces a horizontal surface such as a table. Thus, in some instances a physician may desire to prime the syringe 110 in this way due to the orientation of the syringe 110 as well as the ability to do so with one hand.

There may be other instances during therapy in which the practitioner desires to displace the plunger 120 distally using only one hand. In addition to priming the inflation device 100 as described above, this method of advancing the plunger 120 may also be employed to prime a device connected to the syringe 110, such as a balloon 105.

Without further elaboration, it is believed that one skilled in the art can use the preceding description to utilize the present disclosure to its fullest extent. The examples and embodiments disclosed herein are to be construed as merely illustrative and exemplary, and not a limitation of the scope of the present disclosure in any way. It will be apparent to those having skill in the art that changes may be made to the details of the above-described embodiments without departing from the underlying principles of the disclosure herein. It is intended that the scope of the invention be defined by the claims appended hereto and their equivalents.

1. An inflation device configured to displace fluid, the inflation device comprising:
a syringe body;
a plunger disposed within the syringe body, wherein the plunger is configured displace fluid within the inflation device, and
wherein the plunger is configured to be selectively coupled to the syringe body by a plurality of threads; and
a handle coupled to the plunger, the handle configured to selectively decouple the threads from the syringe body, wherein the handle comprises:
a lever pivotably coupled to the plunger;
a trigger coupled to the lever; and
a sleeve coupled to the lever;
wherein the handle is configured to decouple the threads when a first force is singularly applied to the handle, the first force applied in a distal direction.

2. The inflation device of claim 1, wherein the first force is less than a force required to distally displace the plunger within the syringe body.

3. The inflation device of claim 1, wherein the handle is configured to decouple the threads when a second force is singularly applied to the handle, the second force applied in a proximal direction.

4. The inflation device of claim 3, wherein the second force is greater than the first force.

5. The inflation device of claim 1, wherein the handle further comprises a biasing member configured to apply a distal biasing force to the trigger with respect to the plunger, and wherein decoupling the threads comprises overcoming the biasing force.

6. The inflation device of claim 1, wherein the biasing force is greater than a force required to proximally displace the plunger within the syringe body.

7. The inflation device of claim 1, wherein the lever provides a mechanical advantage to decouple the threads when the first force is applied to the sleeve.

8. The inflation device of claim 7, wherein when the threads are coupled, the lever defines a first factor of mechanical advantage, and when the threads are decoupled, the lever defines a second factor of mechanical advantage, wherein the second factor is greater than the first factor.

9. The inflation device of claim 1, wherein the first force displaces the sleeve relative to the plunger from a first position to a second position decoupling the threads from the syringe body, and
wherein in the first position, the first force is transferred to the plunger through the biasing member, and in the second position, the first force is transferred to the plunger directly through rigid contact between the sleeve and the plunger.

10. An inflation device configured for use in connection with a medical device, the inflation device comprising:
a body component;
a pressurization component configured to increase or decrease pressure within the body component through displacing the pressurization component with respect to the body component;
a coupling mechanism configured to selectively constrain the displacement of the pressurization component with respect to the body component; and
a handle comprising an actuator, the actuator configured to 1) disengage the coupling mechanism upon the application of a single external force applied to the handle, the single external force applied in a distal direction and exceeding a first specified amount, and 2) upon disengagement of the coupling mechanism, distally displace the pressurization component by the application of the single external force exceeding a second specified amount,
wherein the second specified amount is at least partially defined by a friction force between the pressurization component and the body component.

11. The inflation device of claim 10, wherein the second specified amount is less than the first specified amount.

12. The inflation device of claim 10, wherein the actuator is configured to maintain disengagement of the coupling mechanism by the application of the single external force exceeding a third specified amount, and
wherein the third specified amount is less than the second specified amount.

13. The inflation device of claim 12, wherein the third specified amount is less than the first specified amount.

14. The inflation device of claim 12, wherein the third specified amount is less than the friction force.

15. A method of displacing a plunger component of an inflation device, comprising:
obtaining an inflation device comprising:
a syringe body,
a plunger disposed within the syringe body, and
a handle coupled to the plunger, the handle configured to selectively couple and decouple the plunger from the syringe body;
actuating the handle to decouple the plunger from the syringe body by displacing a first component of the handle in a distal direction with respect to the syringe body by externally applying a distally directed force to the first component, the force exceeding a first specified amount; and
displacing the plunger in a distal direction with respect to the syringe body by continued application of the force, the force exceeding a second specified amount less than the first specified amount.

16. The method of claim 15, wherein the handle is actuated by applying the force to the first component by contact between the first component and a static object.

17. The method of claim 16, wherein the user has only one hand in contact with the inflation device.

18. The method of claim 15, further comprising:
actuating the handle to decouple the plunger from the syringe body by squeezing a second component toward the first component such that the second component is displaced toward the first component.

19. The method of claim 18, wherein squeezing the second component toward the first component to decouple the plunger from the syringe body requires a first amount of force applied to the second component, and
wherein holding the second component toward the first component to maintain decoupling of the plunger from the syringe body requires a second amount of force applied to the second component, and
wherein the second amount is less than the first amount.

20. The method of claim 18, further comprising:
displacing the plunger in a proximal direction with respect to the syringe body while maintaining a force, applied to the second component, greater than the second amount and less than the first amount.
